(19) United States
(12) Patent Application Publication
(10) Pub. No.: US 2024/0066245 A1
(43) Pub. Date: Feb. 29, 2024

(54) Device for Collecting Seepage from a Tracheal Incision

(71) Applicant: Nigel Philip Spellman, Longview, TX (US)

(72) Inventor: Nigel Philip Spellman, Longview, TX (US)

(21) Appl. No.: 17/897,717

(22) Filed: Aug. 29, 2022

(51) Int. Cl. A61M 16/04

(52) U.S. Cl. CPC A61M 16/047; A61M 16/0463

(57) ABSTRACT

The present disclosure provides a device for collecting seepage from below a tracheostomy incision on a patient by providing a pad with a groove running transversely to the flow of the seepage. The groove couples to a suction catheter tube which can apply a constant suction to the area of the groove, removing the seepage before it collects on the skin for too long or contaminates a patient's bed. This both increases patient comfort and reduces workload for nursing staff.

### FIELD OF INVENTION

The present invention relates generally to the technical field of medical dressing apparatus. More specifically, the present invention relates to a pad for collecting seepage from below a tracheal incision.

### BACKGROUND

A tracheotomy consists of making an incision in the neck, opening a direct airway to the respiratory system through an incision in the trachea. The resulting opening in the neck and trachea (known as a stoma) serves as a site for a tracheostomy tube to be inserted, allowing ventilators and such to facilitate breathing without the use of the nose or mouth.

Tracheotomy procedures are performed on individuals who, for certain medical reasons, cannot breath on their own or have a difficult time breathing through the normal human respiratory passageways, the nose and mouth. Medical conditions and situations in which a tracheotomy is performed include, but are not limited to, severe facial trauma, head and neck cancers, large congenital tumors of the head and neck (e.g., a bronchial cleft cyst), acute angioedema and inflammation of the head and neck. Tracheotomies also are often utilized in the chronic setting where a patient has a need for long-term mechanical ventilation (e.g., comatose patients).

While a tracheostomy provides a patient who otherwise would have a difficult time breathing with a viable alternative passageway through which they can obtain air, the procedure does have some complications and drawbacks.

In the normal physiological framework in which an individual obtains air through the nose and/or mouth, in addition to functioning to supply air to and from the lungs, the upper airway warms, cleans and moistens air taken into the respiratory pathway. A tracheostomy bypasses these mechanisms. As a result, air entering the respiratory system of an individual through the tracheostomy tube rather than through the upper airway is cooler, dryer and not as clean. In response to these changes to the way the air enters the respiratory system (cooler, drier and dirtier air), the body produces secretions.

Skin care at the tracheotomy is thus one of the key points of nursing. However, patients with tracheotomies often have exudate frequently from the air incision, and cannot be tended often enough to prevent the seepage from causing the patient's skin to become red, swollen, and even eroded, which increases patient suffering and causes nurse-patient disputes; at the same time, nurses need to replace the contaminated bed sheets constantly, increasing the workload of care.

It is within this context that the present invention is provided.

### SUMMARY

The present disclosure provides a device for collecting seepage from below a tracheostomy incision on a patient by providing a pad with a groove running transversely to the flow of the seepage. The groove couples to a suction catheter tube which can apply a constant suction to the area of the groove, removing the seepage before it collects on the skin for too long or contaminates a patient's bed. This both increases patient comfort and reduces workload for nursing staff.

Thus, according to one aspect of the present disclosure there is provided a device for collecting seepage from tracheal incisions, the device comprising a flat elongated pad having an inner surface configured to come into contact with human skin and an opposing second outer protective surface, wherein the inner surface comprises a groove running across the entire length of the pad, the groove being parallel and adjacent to an edge of the pad, the groove having at least one end configured to couple to a suction catheter.

In one embodiment, the device comprises one or more adhering elements configured to secure the device in place against the skin of a patient's chest and neck below a tracheal tube holder with the edge adjacent to the groove being most proximal to the tracheal tube holder.

In an alternative embodiments, the device is a tracheal tube holder comprising a central opening for receiving and coupling to a tracheal tube and slots for receiving ties to secure the inner surface against a tracheal incision on a patient's neck with the device oriented such that the groove rests below the central opening.

In some embodiments, a waterproof foam element covers the inner surface except for the portion of the inner surface with the groove.

In some embodiments, both ends of the groove are configured to couple to a suction catheter.

In some embodiments, the device comprises an integrated suction catheter tube.

### BRIEF DESCRIPTION OF THE DRAWINGS

Various embodiments of the invention are disclosed in the following detailed description and accompanying drawings.

FIG. 1 illustrates an isometric view of the inner surface of a first example configuration of a device according to the present disclosure.

FIG. 2 illustrates an isometric view of the first example configuration being worn by a patient with a tracheal incision.

FIG. 3 illustrates an isometric view of the inner surface of a second example configuration of a device according to the present disclosure.

FIG. 4 illustrates an isometric view of the second example configuration being worn by a patient with a tracheal incision.

Common reference numerals are used throughout the figures and the detailed description to indicate like elements. One skilled in the art will readily recognize that the above figures are examples and that other architectures, modes of operation, orders of operation, and elements/functions can be provided and implemented without departing from the characteristics and features of the invention, as set forth in the claims.

### DETAILED DESCRIPTION AND PREFERRED EMBODIMENT

The following is a detailed description of exemplary embodiments to illustrate the principles of the invention. The embodiments are provided to illustrate aspects of the invention, but the invention is not limited to any embodiment. The scope of the invention encompasses numerous alternatives, modifications and equivalent; it is limited only by the claims.

Numerous specific details are set forth in the following description in order to provide a thorough understanding of the invention. However, the invention may be practiced according to the claims without some or all of these specific details. For the purpose of clarity, technical material that is known in the technical fields related to the invention has not been described in detail so that the invention is not unnecessarily obscured.

The terminology used herein is for the purpose of describing particular embodiments only and is not intended to be limiting of the invention. As used herein, the term “and/or” includes any combinations of one or more of the associated listed items. As used herein, the singular forms “a,” “an,” and “the” are intended to include the plural forms as well as the singular forms, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof.

The present disclosure is based around the concept of providing a device with a groove running along an inner surface that facilitates the application of constant negative pressure to an area where seepage (secretions) from a tracheal incision would otherwise collect, allowing the secretions to be removed as soon as they emerge from the site. This can be done in numerous ways, and two alternative example configurations are provided below.

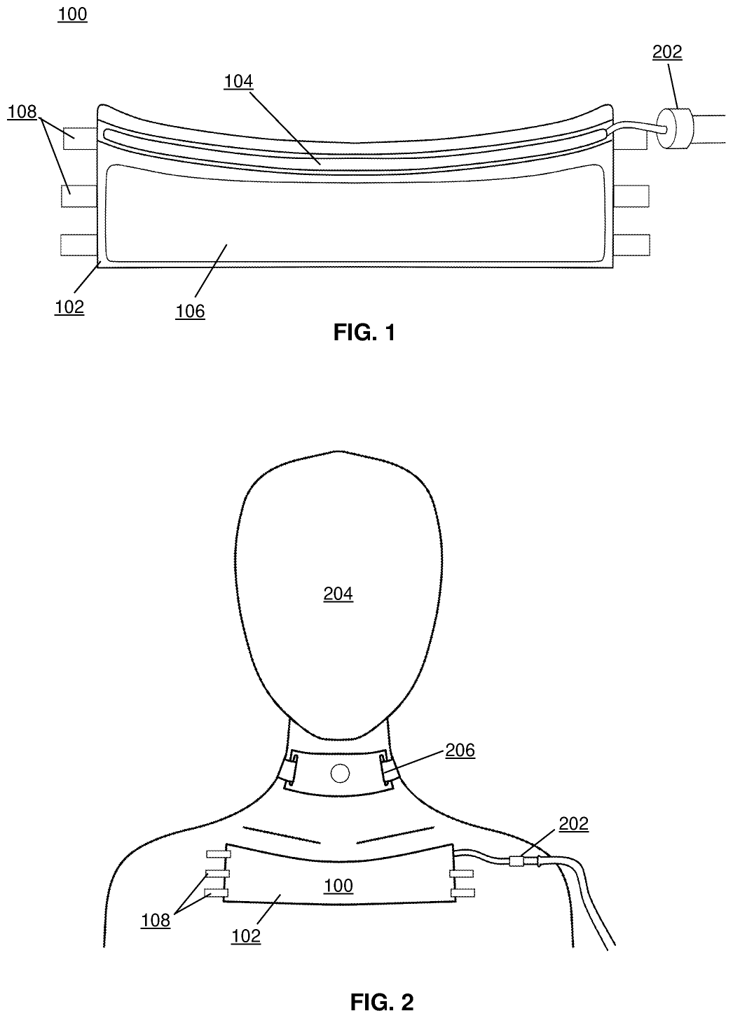

Referring to FIG. 1 and FIG. 2, a first example configuration of such a device is shown with an elongated pad 100 that is configured to be placed on the chest or lower neck of a patient 204 to collect seepage that runs down the neck. FIG. 1 shows a view of the inner surface of the pad 100, and FIG. 2 shows it placed on a patient 204 that has a tracheal tube holder 206 fitted.

The benefit of this example configuration is that it is completely separate from the tracheal tube holder 206, and thus does not require any modifications to the tracheal tube holder.

As can be seen, the pad 102 has a groove 104 running across its length, parallel and adjacent to the edge of the pad which is placed proximal to the tracheal tube holder 206. A plurality of adhesive tape elements 108 secure the pad 102 in place over the patient's skin at a desired placement and orientation. A standard suction catheter tube 202 then couples to one end of the groove 104. The rest of the inner surface of the pad 102 is covered by a waterproof foam element 106 that deals with any seepage that happens to leak down lower.

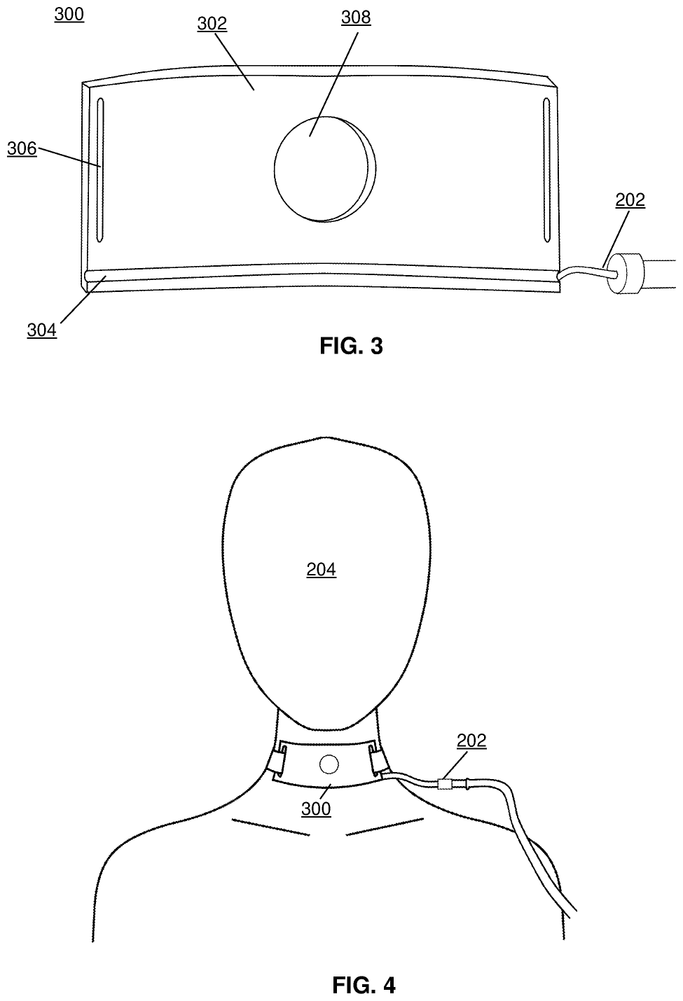

Referring to FIG. 3 and FIG. 4, a second example configuration of the device 300 is shown which integrates with/doubles up as a tracheal tube holder itself, with a groove 304 running along the bottom edge of the device, collecting seepage from just below the incision. FIG. 3 shows a view of the inner surface of the device 300, and FIG. 4 shows it placed on a patient 204.

The benefit of this configuration is that it catches the seepage from the tracheal incision closer to the wound, and therefore reduces skin discomfort to a greater extent.

As can be seen, this configuration of the device 300 is fairly similar to a standard tracheal tube holder, with an inner surface 302 and central opening 308 configured to be placed over the incision in the neck and slots 306 either side for the ties that secure it around the neck. The main difference is the groove 304 running across the bottom edge which is configured to couple to the suction catheter 202.

Unless otherwise defined, all terms (including technical terms) used herein have the same meaning as commonly understood by one having ordinary skill in the art to which this invention belongs. It will be further understood that terms, such as those defined in commonly used dictionaries, should be interpreted as having a meaning that is consistent with their meaning in the context of the relevant art and the present disclosure and will not be interpreted in an idealized or overly formal sense unless expressly so defined herein.

The disclosed embodiments are illustrative, not restrictive. While specific configurations of the device for collecting seepage form a tracheal incision have been described in a specific manner referring to the illustrated embodiments, it is understood that the present invention can be applied to a wide variety of solutions which fit within the scope and spirit of the claims. There are many alternative ways of implementing the invention.

It is to be understood that the embodiments of the invention herein described are merely illustrative of the application of the principles of the invention. Reference herein to details of the illustrated embodiments is not intended to limit the scope of the claims, which themselves recite those features regarded as essential to the invention.

What is claimed is:

1. A device for collecting seepage from tracheal incisions, the device comprising a flat elongated pad having an inner surface configured to come into contact with human skin and an opposing second outer protective surface, wherein the inner surface comprises a groove running across the entire length of the pad, the groove being parallel and adjacent to an edge of the pad, the groove having at least one end configured to couple to a suction catheter.

2. A device according to claim 1, wherein the device comprises one or more adhering elements configured to secure the device in place against the skin of a patient's chest and neck below a tracheal tube holder with the edge adjacent to the groove being most proximal to the tracheal tube holder.

3. A device according to claim 1, wherein the device is a tracheal tube holder comprising a central opening for receiving and coupling to a tracheal tube and slots for receiving ties to secure the inner surface against a tracheal incision on a patient's neck with the device oriented such that the groove rests below the central opening.

4. A device according to claim 1, wherein a waterproof foam element covers the inner surface except for the portion of the inner surface with the groove.

5. A device according to claim 1, wherein both ends of the groove are configured to couple to a suction catheter.

6. A device according to claim 1, wherein the device comprises an integrated suction catheter tube.
